(19) United States
(12) Patent Application Publication
(10) Pub. No.: US 2019/0103188 A1
(43) Pub. Date: Apr. 4, 2019

(54) PATIENT-SPECIFIC CLINICAL DECISION SUPPORT

(71) Applicant: KONINKLIJKE PHILIPS N.V., EINDHOVEN (NL)

(72) Inventors: Saeed Babaeizadeh, Arlington, MA (US); Reza Firoozabadi, Thousand Oaks, CA (US); Eric Helfenbein, Sunnyvale, CA (US)

(21) Appl. No.: 16/086,438

(22) PCT Filed: Mar. 17, 2017
(86) PCT No.: PCT/IB2017/051551

§ 371 Date: Sep. 19, 2018

Related U.S. Application Data
(60) Provisional application No. 62/316,085, filed on Mar. 31, 2016.

(51) Int. Cl. G16H 50/20; G16H 40/20

(52) U.S. Cl. CPC G16H 50/20; G16H 40/20

(57) ABSTRACT

Various embodiments described herein relate to methods and apparatuses for providing patient- specific clinical decision support. Operators such as medical personnel or the like rely on clinical decision support (CDS) procedures to assist in providing healthcare for patients. By activating relevant procedures and deactivating non-relevant procedures for specific patients, operators are less distracted and can more effectively provide healthcare for patients.

### TECHNICAL FIELD

Various embodiments described here generally relate to methods and apparatuses for providing clinical decision support and, more particularly, but not exclusively, to methods and apparatuses for providing patient-specific clinical decision support.

### BACKGROUND

Health institutions and medical personnel have become increasingly interested in more effectively utilizing patient information in conjunction with clinical knowledge to make better health-related decisions. The ultimate goal is to improve the quality of healthcare while lowering the cost of providing healthcare.

There has been a trend to develop fully- or semi-automated methods to intelligently process patient data along with organized clinical knowledge to help care providers make clinical decisions. Such methods in general are referred to as Clinical Decision Support (CDS) procedures. These procedures may provide support for either a specific clinical condition or to a more general health status of a patient.

These procedures may be useful in early recognition of potential hazards and adverse event prevention, thereby reducing clinical errors and improving compliance with quality measures. However, overusing these procedures may be counter-productive and lead to CDS failure. For example, presenting excessive alerts or other notifications may force a clinician to take time out from other tasks. Clinicians may similarly become annoyed with alerts and deactivate the CDS procedures. Or, they may simply ignore or bypass excessive (yet important) alerts.

A need exists, therefore, for CDS methods and apparatuses that overcome the above-mentioned disadvantages.

### SUMMARY

This summary is provided to introduce a selection of concepts in a simplified form that are further described below in the Detailed Description section. This summary is not intended to identify key features or essential features of the claimed subject matter, nor is it intended to be used as an aid in determining the scope of the claimed subject matter.

Various embodiments relate to a method of providing clinical decision support for at least one patient, the method comprising obtaining data that is generally related to the at least one patient; analyzing, using a processor, the obtained data using a plurality of procedures, wherein each of the plurality of procedures has an activation status; and changing, using the processor, the activation status of at least one procedure from deactivated to activated based on the analysis of the obtained data.

These features allow for, for example, patient-specific clinical decision support (CDS) so that medical personnel or other interested parties are only using or are only shown procedures that are relevant for a specific patient.

Various embodiments are described wherein the method further comprises displaying the at least one activated procedure via a user interface. In this embodiment, the method further comprises changing, using the processor, the activation status of the at least one procedure from activated to deactivated; and stopping the display of the deactivated procedure via the user interface.

These features are advantageous because medical personnel or other interested parties are only shown, via the user interface, procedures that are relevant, and are not shown features that are not relevant.

Various embodiments are described wherein the method further comprises changing, using the processor, the activation status of the at least one procedure from activated to deactivated in response to insufficient data related to the at least one patient.

This feature is advantageous because, for example, it conserves computing power by deactivating procedures that are otherwise unable to produce a helpful conclusion due to insufficient information.

Various embodiments are described wherein the method further comprises changing, using the processor, the activation status of the at least one procedure from activated to deactivated by stopping the procedure from analyzing the obtained data.

Stopping a procedure from analyzing obtained data is advantageous because it conserves computing power, for example.

Various embodiments are described wherein the data generally related to the at least one patient includes one or more of patient history, patient demographics, patient symptoms, patient diagnosis, patient vital signs, laboratory data, and patient current health status.

The features of the invention may therefore consider a wide range of data in providing clinical decision support for a patient.

Various embodiments are described wherein the method further comprises issuing a notification to at least one operator regarding the change in activation status of the at least one procedure.

Issuing a notification is advantageous because an operator such as a medical personnel or other interested party is informed regarding which procedure(s) are relevant, and are automatically shown which procedure(s) are activated.

Various embodiments are described wherein the method further comprises issuing a recommendation to change the activation status of the at least one procedure to at least one operator before changing the activation status of the at least one procedure.

Issuing a recommendation to change the activation status of a procedure is advantageous because it informs operator that a particular procedure may be important for a specific patient, and the operator may choose whether or not to activate the particular procedure.

Various embodiments are described wherein the method further comprises, determining, using the processor, a relevancy of the at least one procedure, wherein the processor changes the activation status of the at least one procedure from deactivated to activated based on a determination that the procedure is relevant.

Activating only relevant procedures is advantageous because it conserves computing power and does not distract operators with information regarding non-relevant procedures.

Various embodiments are described wherein the method further comprises, determining, using the processor, a relevancy of the at least one procedure and deactivating the at least one procedure based on a determination that the procedure is not relevant.

Deactivating non-relevant procedures is advantageous because, for example, it conserves computing power and ensures that operators are not distracted by non-relevant procedures.

According to another aspect of the present disclosure, various embodiments relate to an apparatus for providing clinical decision support for at least one patient and a related method and non-transitory machine-readable storage medium, the apparatus comprising: a memory; and a processor in operable communication with the memory, wherein the processor is configured to analyze patient data using a plurality of procedures, wherein each of the plurality of procedures has an activation status; identify a change of the activation status of at least one procedure from deactivated to activated based on the analysis of the at least one procedure; and effect the change of the activation status of at least one procedure from deactivated to activated.

These features allow for, for example, patient-specific clinical decision support (CDS) so that medical personnel or other interested parties are only using or are only shown procedures that are relevant for a specific patient.

Various embodiments are described wherein the apparatus further comprising a user interface for displaying output from the at least one activated procedure. In these embodiments, the processor may be further configured to change the activation status of the at least one procedure from activated to deactivated by stopping the display of the output from the at least one deactivated procedure via the user interface.

These features are advantageous because medical personnel or other interested parties are only shown, via the user interface, procedures that are relevant, and are not shown procedures that are not relevant.

Various embodiments are described wherein the processor is further configured to change the activation status of the at least one procedure from activated to deactivated in response to insufficient data related to the at least one patient.

This feature is advantageous because, for example, it conserves computing power by deactivating procedures that are otherwise unable to produce a helpful conclusion due to insufficient information.

Various embodiments are described wherein the processor is further configured to change the activation status of the at least one procedure from activated to deactivated by stopping the procedure from analyzing the data.

Stopping a procedure from analyzing obtained data is advantageous because it conserves computing power, for example.

Various embodiments are described wherein the patient data includes one or more of patient history, patient demographics, patient symptoms, patient diagnosis, patient vital signs, laboratory data, and patient current health status.

The features of the invention may therefore consider a wide range of data in providing clinical decision support for a patient.

Various embodiments are described wherein in effecting the change of the activation status of at least one procedure from deactivated to activated the processor is configured to automatically activate the at least one procedure. In some embodiments, the processor subsequently issues a notification to at least one operator regarding the change in activation status of the at least one procedure.

Issuing a notification is advantageous because an operator such as a medical personnel or other interested party is informed as to which procedure(s) are relevant for a specific patient, and are automatically shown which procedure(s) are activated.

Various embodiments are described wherein in effecting the change of the activation status of at least one procedure from deactivated to activated the processor is configured to issue a recommendation to change the activation status of the at least one procedure to at least one operator before changing the activation status of the at least one procedure.

Issuing a recommendation to change the activation status of a procedure is advantageous because it informs operator that a particular procedure may be important for a specific patient, and the operator may choose whether or not to activate the particular procedure.

Various embodiments are described wherein the processor is further configured to determine a relevancy of the at least one procedure, wherein the processor changes the activation status of the at least one procedure from deactivated to activated based on a determination that the procedure is relevant.

Activating only relevant procedures is advantageous because it conserves computing power and does not distract operators with information regarding non-relevant procedures.

Various embodiments are described wherein the processor is further configured to determine a relevancy of the at least one procedure and configured to change the activation status of the at least one procedure from activated to deactivated based on a determination that the procedure is not relevant.

Deactivating non-relevant procedures is advantageous because, for example, it conserves computing power and ensures that operators are not distracted by non-relevant procedures.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. Various embodiments will now be described, by way of example, with reference to the accompanying drawings, in which:

FIG. 1 illustrates a patient-specific clinical decision support apparatus in accordance with one embodiment;

FIG. 2 depicts a flowchart of a method of providing clinical decision support in accordance with one embodiment;

FIG. 3 depicts a flowchart of a method of providing clinical decision support in accordance with another embodiment;

FIG. 4 depicts a flowchart of a method of providing clinical decision support in accordance with another embodiment;

FIG. 5 depicts a flowchart of a method of providing clinical decision support in accordance with another embodiment;

FIG. 6 depicts a flowchart of a method of providing clinical decision support in accordance with another embodiment;

FIG. 7 depicts a flowchart of a method of providing clinical decision support in accordance with another embodiment;

FIG. 8 depicts a flowchart of a method of providing clinical decision support in accordance with another embodiment;

FIG. 9 depicts a flowchart of a method for providing clinical decision support with respect to Acute Kidney Injury in accordance with one embodiment;

FIG. 10 illustrates a table showing exemplary parameters that may be considered when implementing the method of FIG. 9;

FIG. 11 depicts a flowchart of a method for providing clinical decision support with respect to Acute Kidney Injury in accordance with another embodiment;

FIG. 12 illustrates a table showing exemplary parameters that may be considered when implementing the method of FIG. 11;

FIG. 13 depicts a flowchart of a method for providing clinical decision support with respect to Acute Kidney Injury in accordance with another embodiment;

FIG. 14 illustrates a table showing exemplary parameters that may be considered when implementing the method of FIG. 13;

FIG. 15 depicts a flowchart of a method for providing clinical decision support with respect to Acute Respiratory Distress Syndrome (ARDS) in accordance with one embodiment;

FIG. 16 illustrates a table showing exemplary parameters that may be considered when implementing the method of FIG. 15; and

FIG. 17 presents a system for providing clinical decision support in accordance with one embodiment.

### DETAILED DESCRIPTION

Various embodiments are described more fully below with reference to the accompanying drawings, which form a part hereof, and which show specific exemplary embodiments. However, the concepts of the present disclosure may be implemented in many different forms and should not be construed as limited to the embodiments set forth herein; rather, these embodiments are provided as part of a thorough and complete disclosure, to fully convey the scope of the concepts, techniques and implementations of the present disclosure to those skilled in the art. Embodiments may be practiced as methods, systems or devices. Accordingly, embodiments may take the form of a hardware implementation, an entirely software implementation (running on appropriate supporting hardware such as a processor) or an implementation combining software and hardware aspects. The following detailed description is, therefore, not to be taken in a limiting sense.

Reference in the specification to “one embodiment” or to “an embodiment” means that a particular feature, structure, or characteristic described in connection with the embodiments is included in at least one example implementation or technique in accordance with the present disclosure. The appearances of the phrase “in one embodiment” in various places in the specification are not necessarily all referring to the same embodiment.

Some portions of the description that follow are presented in terms of symbolic representations of operations on data stored within a computer memory. These descriptions and representations are used by those skilled in the data processing arts to most effectively convey the substance of their work to others skilled in the art. Such operations typically require physical manipulations of physical quantities. Usually, though not necessarily, these quantities take the form of electrical, magnetic or optical data capable of being stored, transferred, combined, compared and otherwise manipulated. It is convenient at times, principally for reasons of common usage, to refer to such data as bits, values, elements, symbols, characters, terms, numbers, or the like. Furthermore, it is also convenient at times, to refer to certain arrangements of steps requiring physical manipulations of physical quantities as modules or code devices, without loss of generality.

However, all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system memories or registers or other such information storage, transmission or display devices. Portions of the present disclosure include processes and instructions that may be embodied in software, firmware or hardware, and when embodied in software, may be downloaded to reside on and be operated from different platforms used by a variety of operating systems.

The present disclosure also relates to an apparatus for performing the operations herein. This apparatus may be specially constructed for the required purposes, or it may comprise a general-purpose computer selectively activated or reconfigured by a computer program stored in the computer. Such a computer program may be stored in a non-transitory computer readable storage medium, such as, but is not limited to, any type of disk including floppy disks, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each may be coupled to a computer system bus. Furthermore, the computers referred to in the specification may include a single processor or may be architectures employing multiple processor designs for increased computing capability.

The processes and displays presented herein are not inherently related to any particular computer or other apparatus. Various general-purpose systems may also be used with programs in accordance with the teachings herein, or it may prove convenient to construct more specialized apparatus to perform one or more method steps. The structure for a variety of these systems is discussed in the description below. In addition, any particular programming language that is sufficient for achieving the techniques and implementations of the present disclosure may be used. A variety of programming languages may be used to implement the present disclosure as discussed herein.

In addition, the language used in the specification has been principally selected for readability and instructional purposes and may not have been selected to delineate or circumscribe the disclosed subject matter. Accordingly, the present disclosure is intended to be illustrative, and not limiting, of the scope of the concepts discussed herein.

A clinical system, e.g., electronic health records (EHR) or patient monitoring system, may have several CDS procedures implemented. It may be neither practical nor necessary to activate all procedures for every patient. It may instead be desirable to know which procedures are most useful for monitoring a particular patient to support patient-specific clinical decisions. The present application describes methods and apparatuses tasked to recommend specific CDS procedures based on, for example, patient demographics, clinical data, admission info, and other relevant parameters. The features of the present invention may be implemented in medical support (software) such as clinical chartings, electronic health records, and central monitoring stations.

Although the features of the invention are described as being implemented in healthcare applications, it is contemplated that they may be implemented in other applications. For example, the features of the invention may be used in financial market analysis, manufacturing operations, logistic operations, climate analysis, counter-terrorism/crime prevention, or any other application that relies on the analysis of large amounts of data.

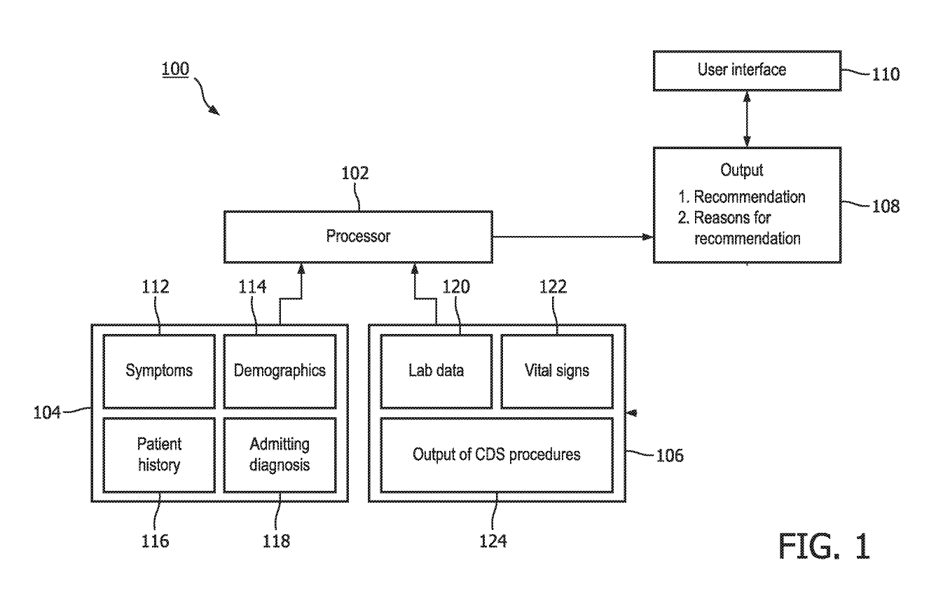

FIG. 1 schematically illustrates a patient-specific clinical decision support (CDS) apparatus 100 in accordance with one embodiment. The apparatus 100 may include a processor 102, a set of static parameters 104, and a set of dynamic parameters 106 to produce an output 108 that may be presented on a user interface 110.

In use, static parameters 104 and dynamic parameters 106 may be provided to the processor 102. The static parameters 104 may include relatively constant information relating to a patient that was obtained, e.g,. at the time of check-in, and the dynamic parameters 106 may include changing information obtained intermittently or continuously and in at least substantially real time or with some delay that does not diminish the value of the dynamic parameters 106.

The parameters 104 and 106 may be analyzed by the processor 102 to produce an output 108 regarding which procedures should be activated or deactivated. The output 108 may be shown via the user interface 110. The output 108 may, for example, be a recommendation to activate a procedure used to detect acute kidney injury as well as reasons for recommending the activation. Or, a certain procedure may automatically be activated or deactivated, and an optional notification to that effect may be provided to an operator.

In the context of this application, the term “procedure” may refer to any process used to predict, detect, and/or monitor a certain patient condition. For example, as discussed in greater detail below, there may be a procedure to predict, detect, and monitor Acute Kidney Injury (AKI) and a procedure to predict, detect, and monitor Acute Respiratory Distress Syndrome (ARDS). As yet another example, Applicants have developed an Early Detection Index (EDI) for the identification of patients with impending critical illness that uses vital signs in scoring functions to determine the probability of deterioration.

In the context of this application, the term “activation status” may refer to whether a procedure is activated or deactivated. The term “activated,” as it applies to a procedure, may mean an output from the procedure is presented visibly on a display. Or, a procedure may be activated and its output suppressed, i.e., not displayed, but it may still communicate updates regarding a patient to an operator such as a medical personnel, care provider, or other interested party (hereinafter “operator”).

The term “deactivated,” as it applies to a procedure, may mean the procedure is being executed with the results of that execution not presented on a display. Additionally or alternatively, it may mean the procedure is configured to not communicate updates, or it may mean the procedure is not running at all.

The features of the invention may therefore only visually present a procedure if/when it is relevant. For example, assume a certain procedure relies on information relating to a patient's respiratory rate, but there is no respiratory rate information available for that patient (e.g., the patient's respiratory rate is not being measured or the relevant sensor device has malfunctioned). There may be no reason for this procedure to be visually presented to an operator because it may produce meaningless conclusions regarding the patient. Therefore, the output 108 may be the automatic deactivation of this procedure, or at least a recommendation to deactivate the procedure.

As another example, assume a procedure is running silently in the background and is not visually presented to an operator. If the procedure detects (e.g., based on a laboratory result) a patient may have a certain condition warranting further medical attention, the output 108 may be to automatically present the procedure on a display so an operator can more easily monitor the procedure. Or, the output 108 may be a recommendation to the operator to elect to display the procedure, along with accompanying reasons.

The processor 102 may be any hardware device capable of processing the static parameters 104 and the dynamic parameters 106. The processor 102 may include a microprocessor, a field programmable gate array (FPGA), application-specific integrated circuit (ASIC), or other similar device. In some embodiments, such as those relying on one or more ASICs, the functionality described as being provided in part via software may instead be hardwired into the operation of the ASICs, and, as such, the associated software may be omitted.

The processor 102 may be pre-programmed with certain procedures. Alternatively, the procedures executed by the processor 102 may be added and removed based on the varying needs of a particular patient and/or operator.

The static parameters 104 may include relatively unchanging information relating to a patient that is obtained at the time of patient admission or is otherwise previously known. Exemplary static parameters 104 may include information relating to patient symptoms 112, demographics 114, patient history 116, admitting diagnosis 118, and other information relating to the patient's current health status.

The symptoms 112 may be described by the patient to medical personnel at the time of patient check-in. The symptoms 112 may also include any characteristics of the patient observed by medical personnel.

Demographics 114 may include information previously known by the healthcare institution or medical personnel, or information obtained at the time of patient admission. The demographics may include, for example, patient age, gender, height, weight, body mass index, etc.

Patient history 116 may include information previously known by the healthcare institution, or information obtained at the time of patient admission. Patient history 116 may include information such as previous patient diagnoses, patient family history, allergies, whether the patient has traveled abroad recently, etc.

Patient admitting diagnosis 118 may be the initial diagnosis at the time of admission and may be based on at least the symptoms 112, demographics 114, and patient history 116. The diagnosis 118 may be made by medical personnel such as a doctor, nurse, physician, or the like.

The dynamic parameters 106 may include information that tends to change with time and is obtained intermittently or continuously and at least substantially in real time or with some delay that does not diminish the value of the dynamic parameters 106. Exemplary dynamic parameters 106 may include lab data 120, vital signs 122, and the output of other CDS procedures 124.

Laboratory data 120 may include the results of certain tests performed on the patient. The laboratory data 120 obtained may depend on the test(s) performed, which may in turn vary depending on the patient and their health status.

The vital signs 122 may be obtained autonomously or by medical personnel. The vital signs 122 may be gathered via appropriate sensor devices and may include arterial (systolic) blood pressure, heart rate, respiratory rate, temperature, O₂ saturation (e.g., arterial oxygen saturation (SpO₂), or the like. The vital signs 122 gathered may vary and may depend on the particular patient and their health status.

The output of other CDS procedures 124 may be analyzed as well. For example, if a procedure used to monitor Acute Kidney Injury is activated and detects (or outputs a value that is or may be indicative of) another health-related condition (e.g., an illness), the condition may be used to make recommendations of additional CDS procedures to activate. As another example, the output of the AKI procedure may be sufficiently low as to indicate that the AKI procedure itself is less relevant and should be deactivated. Thereafter, if the AKI procedure continues to run in its deactivated state (e.g., in the background, or on a less frequent basis) and the output begins to rise to a relevant level, the AKI procedure may be activated (or recommended or activation).

The output 108 may be presented to an operator such as medical personnel or the like via any suitable user interface 110 with a display. For example, the user interface 110 may be a PC, laptop, smartphone, tablet, or the like. The user interface 110 may display procedures as well as other information in the form of tables, graphs, color schemes, or in any other format convenient for an operator. The user interface 110 may receive information relating to the patient via any wireless or hardwired connection.

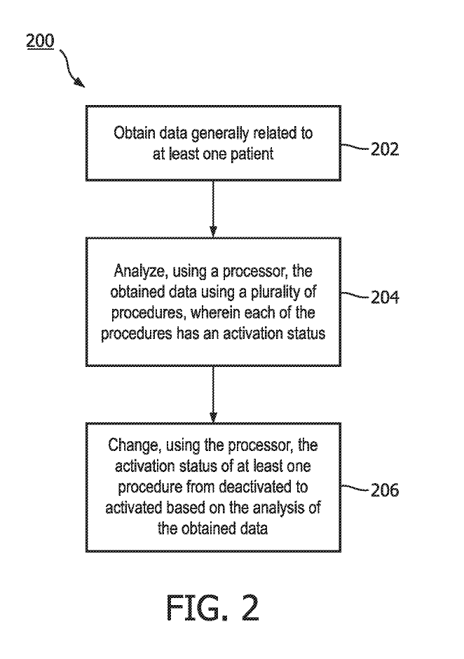

FIG. 2 depicts a flowchart of a method 200 of providing clinical decision support (CDS) for at least one patient. Step 202 involves obtaining data generally related to at least one patient. The obtained data may include any or all of the static parameters 104 and dynamic parameters 106 described previously.

Step 204 involves analyzing the obtained data using a plurality of procedures, wherein each procedure has an activation status. As stated previously, “activation status” may refer to whether a procedure is activated or deactivated.

These procedures may relate to a variety of patient conditions and/or illnesses. The procedures used may be pre-set, such that when any patient is admitted there is initially a set of predetermined procedures that analyze patient data. Or, the procedures used may be tailored to a specific patient based on the static parameters, for example.

In this embodiment, the procedures may initially be deactivated (e.g., not visually presented to an operator and/or configured to not communicate any updates to an operator). Step 206, however, involves changing the activation status of at least one procedure from deactivated to activated based on the analysis of the obtained data.

In this embodiment, for example, changing the activation status of a procedure may include automatically changing the activation status (e.g., by automatically presenting the procedure via a display). Or, changing the activation status may mean issuing a recommendation to an operator to change the activation status of a procedure so that the procedure is visually presented via a display, and can therefore be followed more easily by an operator.

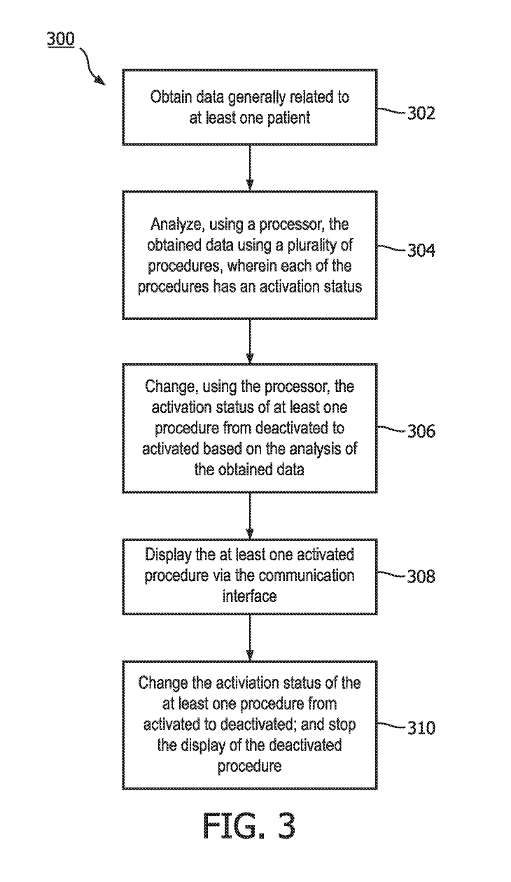

FIG. 3 depicts a flowchart of a method 300 of providing clinical decision support in accordance with another embodiment. Steps 302, 304, and 306 are similar to steps 202, 204, and 206 of FIG. 2, respectively, and are not repeated here.

Step 308 involves displaying the at least one activated procedure via a user interface. The user interface may be the user interface 110 of FIG. 1, for example. The procedure may be displayed graphically or tabularly, for example.

Step 310 involves changing the activation status of the at least one procedure from activated to deactivated; and stopping the display of the deactivated procedure. By not presenting the deactivated procedure, an operator is not distracted by the deactivated procedure and the display of the user interface 110 may be less cluttered, for example.

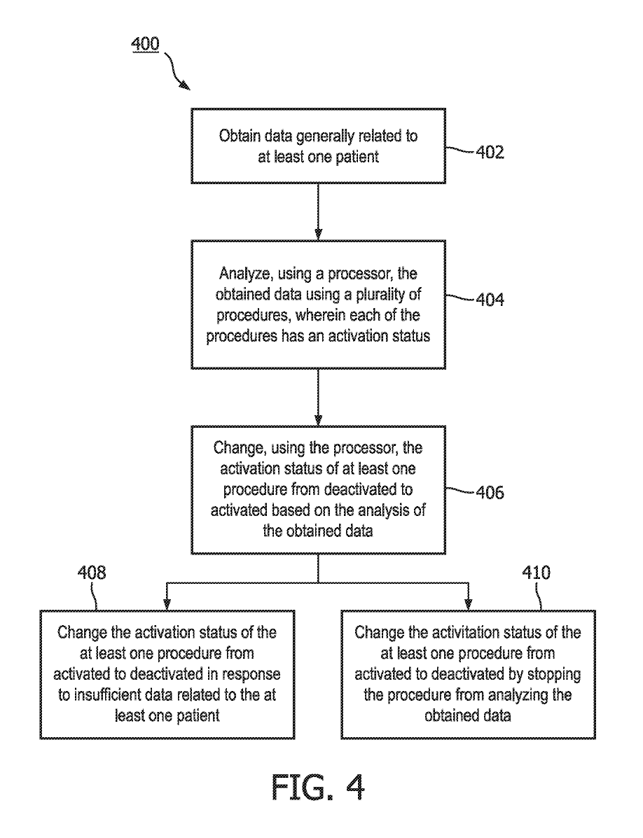

FIG. 4 depicts a flowchart of a method 400 of providing clinical decision support (CDS) in accordance with another embodiment. Steps 402, 404, and 406 are similar to steps 202, 204, and 206 of FIG. 2, respectively, and are not repeated here. Step 408 involves changing the activation status of the at least one procedure from activated to deactivated in response to insufficient data related to the at least one patient. As stated previously, there may be no reason to have a procedure running if it cannot produce a meaningful result due to missing or incomplete data. Therefore, computing resources are preserved.

Step 410 involves changing the activation status of the at least one procedure from activated to deactivated by stopping the procedure from analyzing the obtained data. Step 410 is distinguishable from step 310 of FIG. 3, in that rather than just stopping the display of the procedure as in step 310, the procedure is stopped from analyzing the data entirely in step 410.

It is noted that steps 408 and 410 may be performed concurrently. In other words, step 408 merely describes a reason for deactivating a procedure (i.e., insufficient data), while step 410 describes what is done to deactivate the procedure (i.e., in this embodiment, to “deactivate” means to stop the procedure from analyzing data all together). As another example, a procedure may be deactivated due to insufficient data (such as in step 408), but the deactivation may only stop the display of the procedure (as in step 310).

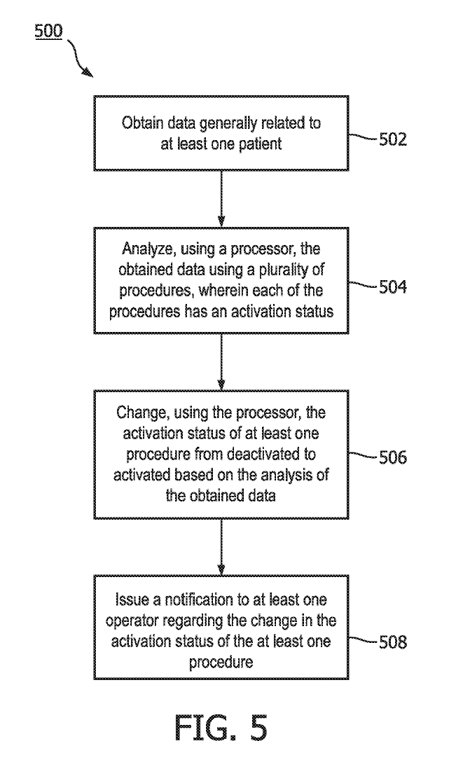

FIG. 5 depicts a flowchart of a method 500 of providing clinical decision support in accordance with another embodiment. Steps 502, 504, and 506 are similar to steps 202, 204, and 206 of FIG. 2, respectively, and are not repeated here.

Operators may wish to know when (and why) activation statuses of procedures change so they remain informed about a patient's treatment. Step 508 therefore involves issuing a notification to at least one operator regarding the change in the activation status of the at least one procedure. In this embodiment, the activation status may automatically be changed, and a notification to that effect may be issued to an operator such as medical personnel or the like. This notification may be presented via the user interface 110, and may be in the form of a visual message (e.g., a text message), an audio message, a tactile (e.g., haptic-based) message, or some combination thereof. The notification may include the reason(s) why the activation status of a procedure was changed.

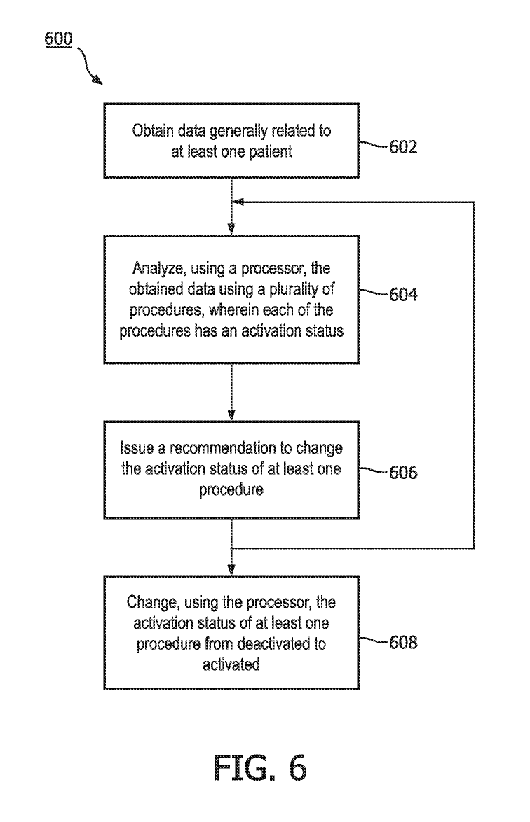

FIG. 6 depicts a flowchart of a method 600 of providing clinical decision support (CDS) in accordance with another embodiment. Steps 602 and 604 are similar to steps 202 and 204 of FIG. 2, respectively, and are not repeated here.

Operators may ultimately want to have a say in whether a procedure's activation status is changed or not. Step 606, therefore, involves issuing a recommendation to change the activation status of at least one procedure to an operator. This recommendation may be presented via the user interface 110, and may be in the form of a visual message (e.g., a text message), an audio message, a tactile (e.g., haptic-based) message, or some combination thereof.

An operator may review the recommendation as well as the particular reason(s) for the recommendation. Based on the operator's personal knowledge and medical expertise, he or she may elect to agree to the recommendation or ignore the recommendation.

For example, the user interface 110 may present virtual icons designated with labels “Change Activation Status” or “Dismiss Recommendation without Changing Activation Status.” An operator may use a mouse-cursor device or a stylus device to select the appropriate label, or may simply touch the appropriate icon (if the user interface 110 is configured with a touch-screen, for example). It is noted that these labels, icons, and methods of electing appropriate icons are merely exemplary.

If the operator elects the “Change Activation Status” option, the method 600 may then proceed to step 608 which involves changing the activation status of the at least one procedure from deactivated to activated. If the operator elects to dismiss the recommendation so that the activation status is not changed, the method 600 may return to step 604 and continue to analyze the obtained data.

It is also contemplated than an operator may input additional instructions so that he or she is not bothered by a similar recommendation in the future. The operator may, for example, input instructions via the user interface 100 to only issue a recommendation if the patient's blood pressure reaches a certain level, for example.

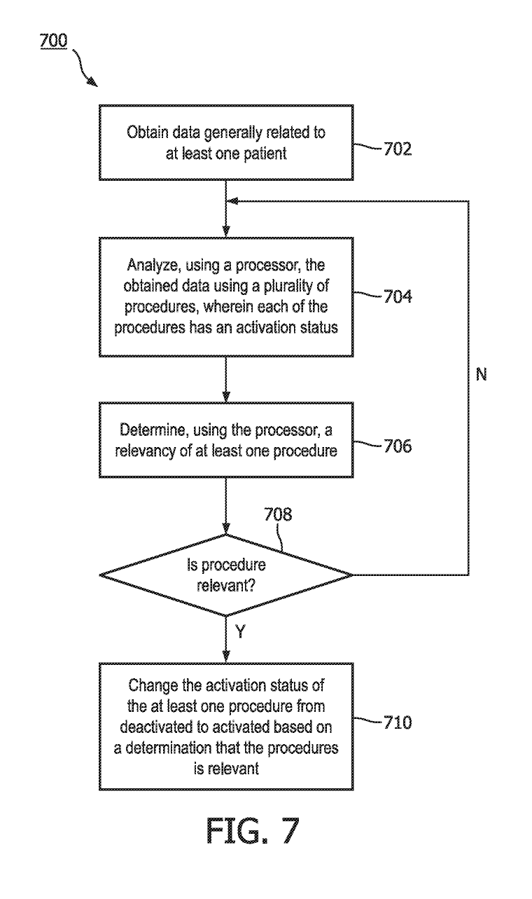

FIG. 7 depicts a flowchart of a method 700 of providing clinical decision support (CDS) in accordance with another embodiment. Steps 702 and 704 are similar to steps 202 and 204 of FIG. 2, respectively, and are not repeated here.

Step 706 involves determining, using the processor, a relevancy of at least one procedure. In the context of the present application, the term “relevant” may simply mean that the processor has received data that indicates a patient is at least at risk of developing a condition. More specifically, a procedure may be “relevant” if the benefit of being activated outweighs any disadvantages of being activated that may result from, for example, updates sent to an operator regarding a patient's health status.

A procedure will likely not be relevant unless it is applicable for a certain patient. For example, the AKI procedure would be relevant for a patient with acute kidney injury. However, it may not be applicable to this patient if their urine output isn't being measured. In this scenario, the procedure may remain deactivated (but a recommendation may be issued to start measuring the patient's urine output).

Step 708 considers whether a particular procedure is relevant or not. If the procedure is relevant, then the method 700 may proceed to step 710 which involves changing the activation status of the procedure from deactivated to activated based on a determination that the procedure is relevant. If, on the other hand, the procedure is not relevant, the method 700 may return back to step 704 to continue to analyze the obtained data.

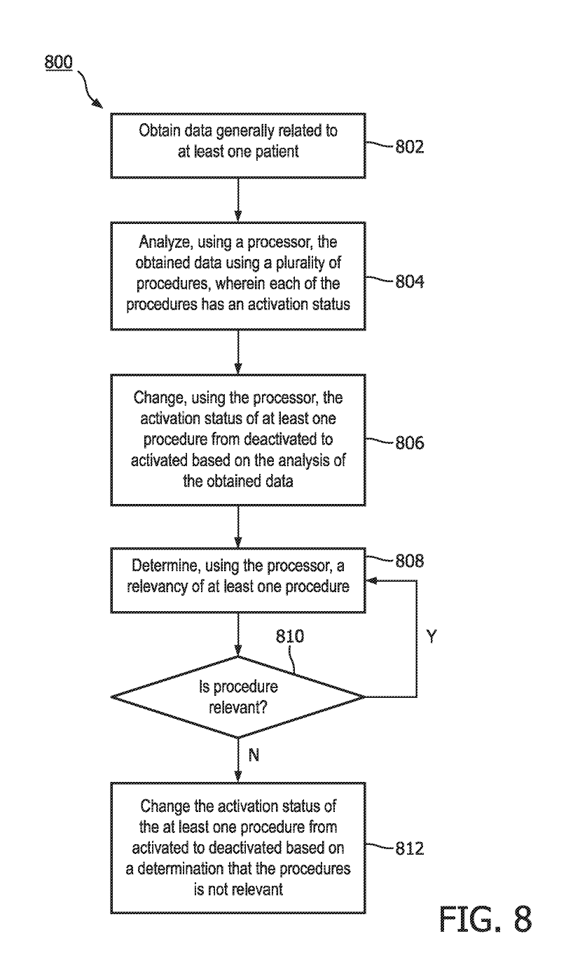

FIG. 8 depicts a flowchart of a method 800 of providing clinical decision support for at least one patient in accordance with another embodiment. Steps 802, 804, and 806 are similar to steps 202, 204, and 206 of FIG. 2, respectively, and are not repeated here.

Step 808 involves determining, using the processor, a relevancy of at least one procedure. A procedure may be “relevant” if it satisfies the requirements described above.

Step 810 considers whether the particular procedure is relevant. If a particular procedure is determined to not be relevant (e.g., it is not applicable due to insufficient data, it does not indicate a patient is at risk of a particular health condition, or other procedures take priority), then method 800 may proceed to step 812. Step 812 involves changing the activation status of the at least one procedure from activated to deactivated based on a determination that the procedure is not relevant. The procedure may be deactivated in any of the ways discussed previously.

If the procedure is determined to be relevant, then method 800 may return to step 808, and the procedure may remain activated. The method 800 may continue to determine whether a particular procedure is relevant based on the analysis of the patient data.

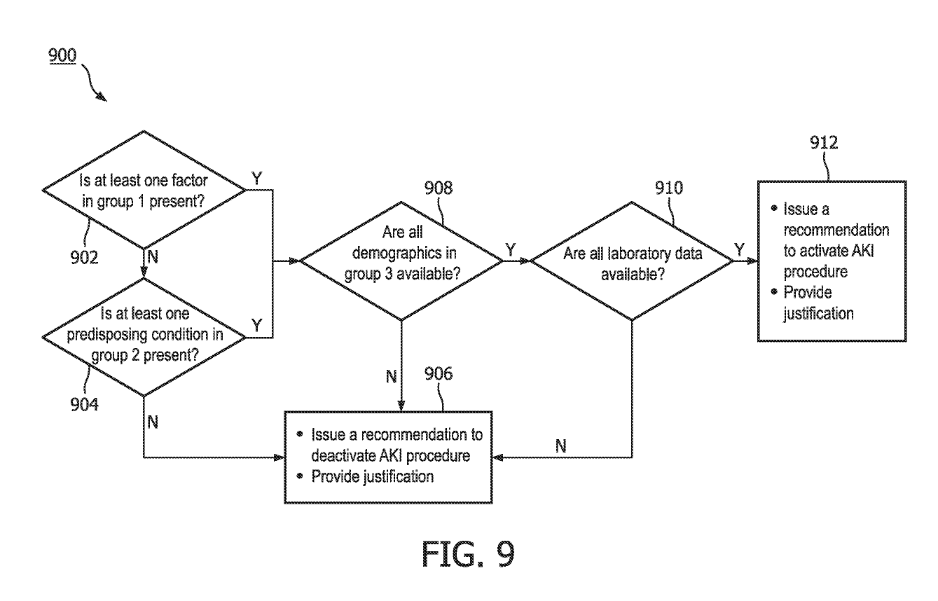

FIG. 9 depicts a flowchart of an exemplary method 900 of providing clinical decision support in the context of monitoring Acute Kidney Injury. Acute Kidney Injury affects several hundred thousands of patients a year and is commonly fatal. Despite increasing incidence and high mortality, very few preventive measures are used to reduce the likelihood of AM. Most frequently, AKI is not present at the time of patient admission but develops over a period of hours to days in subsets of patients with predisposing conditions such as sepsis, trauma and shock and corresponding medical and surgical interventions.

The diagnosis of AKI uses indications of increased serum creatinine or decreased urine output. Serum creatinine is the most widely used test for the diagnosis of AKI. However, serum creatinine has several limitations. First, serum creatinine is itself a late marker of disease and thus does not allow for early recognition of AKI. Second, even small changes in serum creatinine are associated with significant increases in mortality. Last, minimal rise in serum creatinine (≥0.3 mg/dL) is often ignored by many clinicians. Urine output provides a more sensitive AKI detection than serum creatinine, but is not widely used due to poor urine output documentation, use of diuretics, and limited use of Foley catheter.

Thus, the method 900 of FIG. 9 addresses several of the limitations described above. Method 900 may use patient information and lab results such as urine output and/or serum creatinine to recommend activating an AKI procedure. Without the method 900 (or a similar method for activating or recommending activation of the AKI procedure), the AKI procedure may not be activated by medical personnel for patients who, for example, are admitted to a healthcare institution because of trauma.

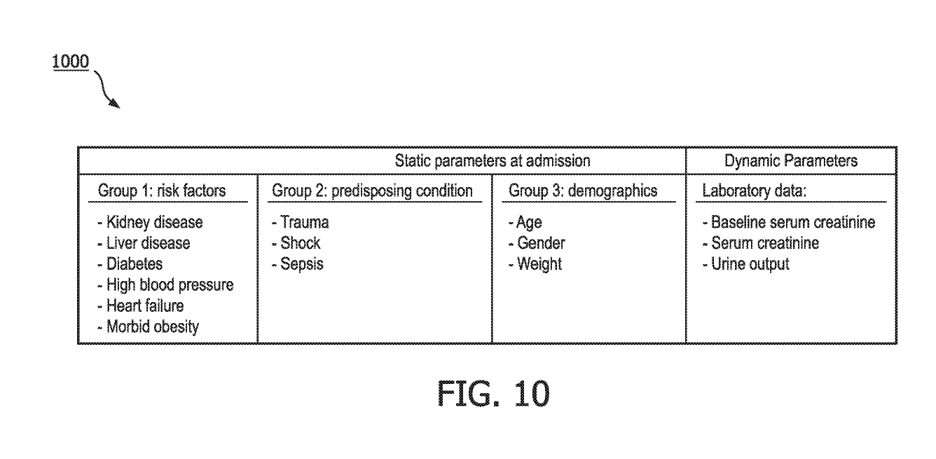

FIG. 9 and method 900 are to be reviewed in conjunction with the table 1000 of FIG. 10. Table 1000 lists exemplary parameters that may be considered when implementing the method 900 of FIG. 9.

Step 902 of method 900 considers whether there is at least one factor in Group 1 (of the table 1000 of FIG. 10) present. Group 1 of 1000 lists several risk factors for AKI that may be obtained at the time of patient admission. These factors include, but are not necessarily limited to, kidney disease, liver disease, diabetes, high blood pressure, heart failure, and morbid obesity.

If there is not at least one factor in Group 1 present, the method 900 may proceed to step 904. Step 904 considers whether there is at least one predisposing condition in Group 2 of table 1000 present. Group 2 of table 1000 lists conditions mentioned previously and include, but are not limited to, trauma, shock, and sepsis.

If there is neither at least one factor from Group 1 present, nor at least one predisposing condition in Group 2 present, the method 900 may proceed to step 906. Step 906 involves issuing a recommendation to deactivate the AKI procedure, along with a justification for recommending deactivating the AKI procedure. For example, the AKI procedure may not be applicable due to insufficient data and/or none of the conditions indicative of AKI are present.

If, on the other hand, at least one factor in Group 1 is present, at least one predisposing condition in Group 2 is present, or both at least one factor in Group 1 and at least one predisposing condition in Group 2 are present, method 900 may proceed to step 908. Step 908 considers whether all demographics in Group 3 of table 1000 are available. The demographics in Group 3 of table 1000 include, but may not necessarily be limited to, patient age, gender, and weight. If not all demographics in Group 3 are available, the method may proceed to 906 to issue a recommendation to deactivate the AKI procedure as discussed previously.

If all demographics in Group 3 are available, the method 900 may proceed to step 910. Step 910 considers whether all laboratory data is available. The laboratory data are considered dynamic parameters and include, but are not necessarily limited to baseline serum creatinine, and urine output. If not all laboratory data is available, the method 900 may proceed to step 906 to issue a recommendation to deactivate the AKI procedure as discussed previously.

If all laboratory data is available, the method 900 may proceed to step 912. Step 912 involves issuing a recommendation to activate the AKI procedure, along with a justification for recommending activating the AKI procedure. As can be deduced from steps 902, 904, 908, and 910 of method 900, the recommendation to activate the AKI procedure may be issued because at least one risk factor is present, at least one predisposing condition is present, all demographics are available, and all laboratory data is available.

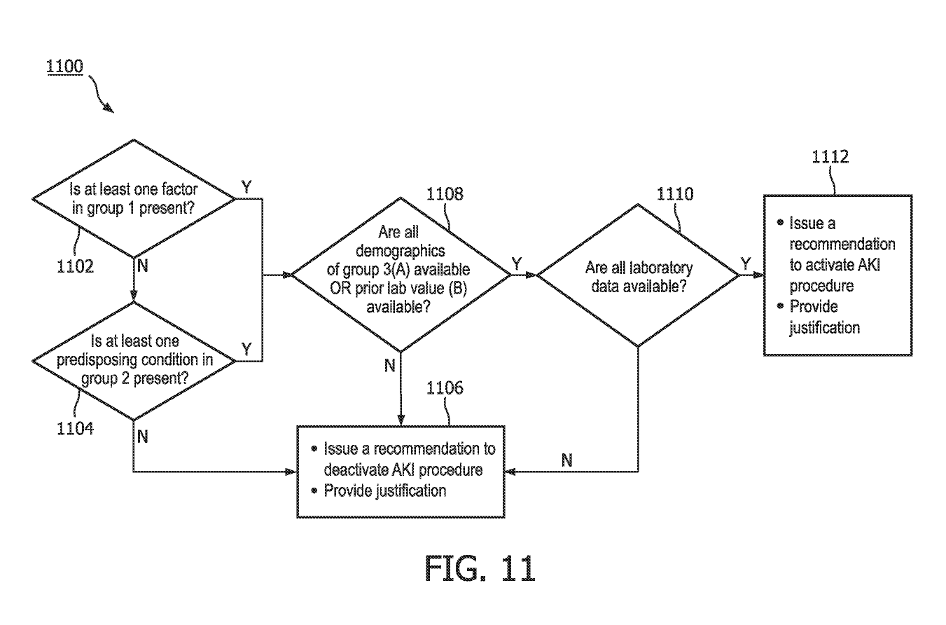

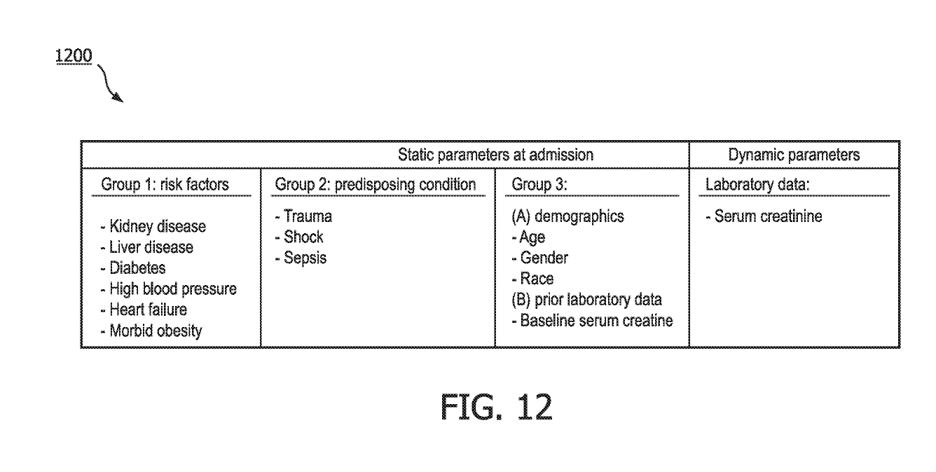

FIG. 11 depicts a flowchart of a method 1100 of providing clinical decision support in the context of monitoring AKI based specifically on lab results of Serum Creatinine (SCr). FIG. 11 and method 1100 are to be reviewed in conjunction with table 1200 of FIG. 12.

Method 1100 and table 1200 are similar to method 900 and table 1000, respectively. However, the method 1100 is based specifically on lab results of Serum Creatinine. This method 1100 relies on a current value of serum creatinine (i.e., the dynamic parameter in table 1200) and either (i) age, gender, and race; or (ii) a baseline serum creatinine value.

Accordingly, step 1108 of method 1100 considers whether all demographics of Group 3(A) (age, gender, race) are available or whether there is a baseline serum creatinine value available. If neither all demographics are available, nor a baseline serum creatinine value is available, method 1100 may proceed to step 1106 to issue a recommendation deactivate this AKI procedure (similar to step 906 of FIG. 9).

If, on the other hand, all demographics are available and/or there is a baseline serum creatinine value available, method 1100 may proceed to step 1110. Step 1110 considers whether all current laboratory data (in this case, serum creatinine) is available. If there is no current data for serum creatinine, the method 1100 may proceed to step 1106 discussed previously.

If, on the other hand, serum creatinine data is available, method 1100 may proceed to step 1112 to issue a recommendation to activate this AKI procedure (similar to step 912 of FIG. 9).

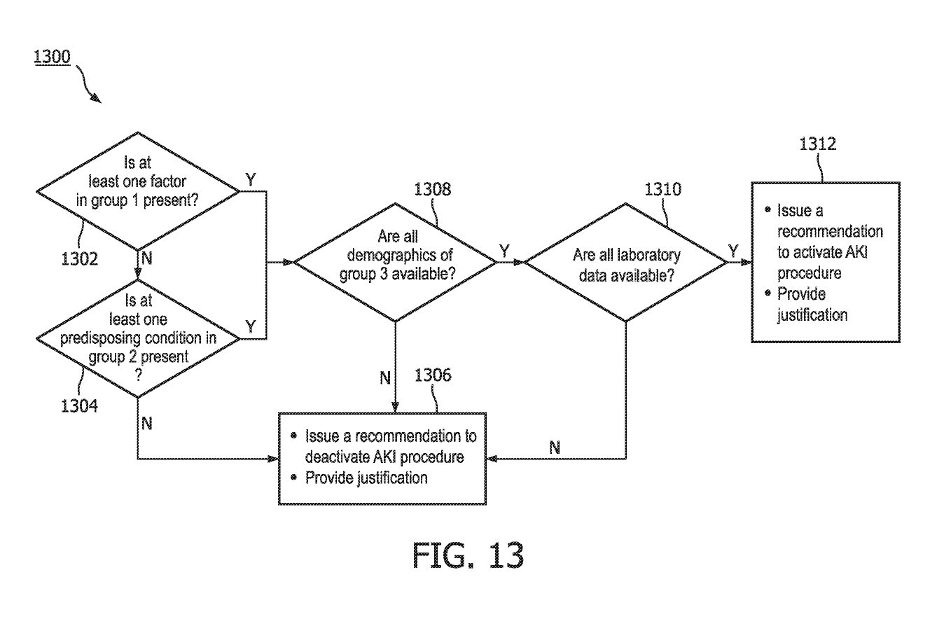

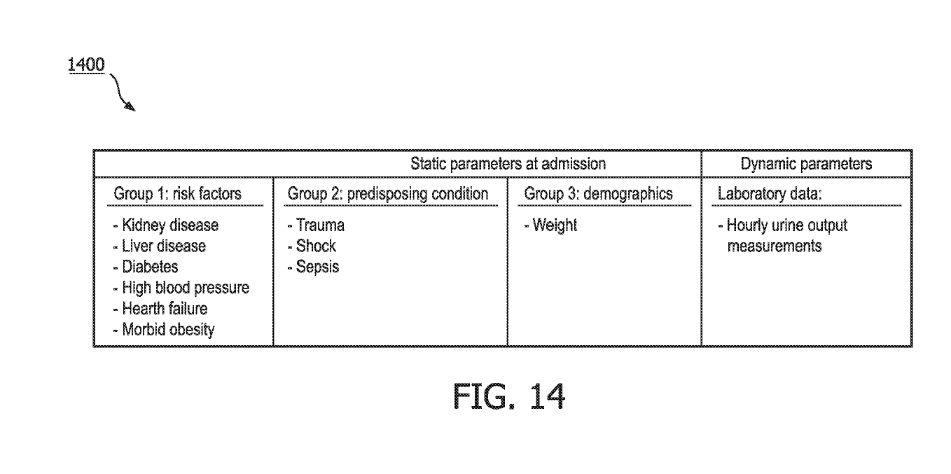

FIG. 13 depicts a flowchart of a method 1300 of providing clinical decision support in the context of monitoring AKI based on urine output of a patient. FIG. 13 and method 1300 are to be reviewed in conjunction with table 1400 of FIG. 14.

Method 1300 and table 1400 are similar to method 900 and table 1000, respectively. However, method 1300 is based specifically on urine output of a patient. This method 1300 relies on knowledge of a patient's weight and measurements of the patient's urine output, taken hourly.

Accordingly, step 1308 considers whether all demographics of Group 3 (weight, in this case) are available. If the patient's weight is not available, method 1300 may proceed to step 1306 to issue a recommendation to deactivate the AKI procedure (similar to step 906 of FIG. 9).

If, on the other hand, the patient's weight is available, method 1300 may proceed to step 1310. Step 1310 considers whether all laboratory data is available. In this method, the required laboratory data is hourly urine output measurements. If this data is not available, the method 1300 may proceed to step 1306 discussed previously.

If, on the other hand, hourly urine output measurements are available, method 1300 may proceed to step 1312 to issue a recommendation to activate this AKI procedure (similar to step 912 of FIG. 9).

Methods 1100 and 1300 may be performed simultaneously. The severity of AKI may be based on the maximum severity as indicated by either method 1100 or 1300. Or, the severity of AKI may be indicated by the result of just one of method 1100 or 1300 if the other does not have the required values.

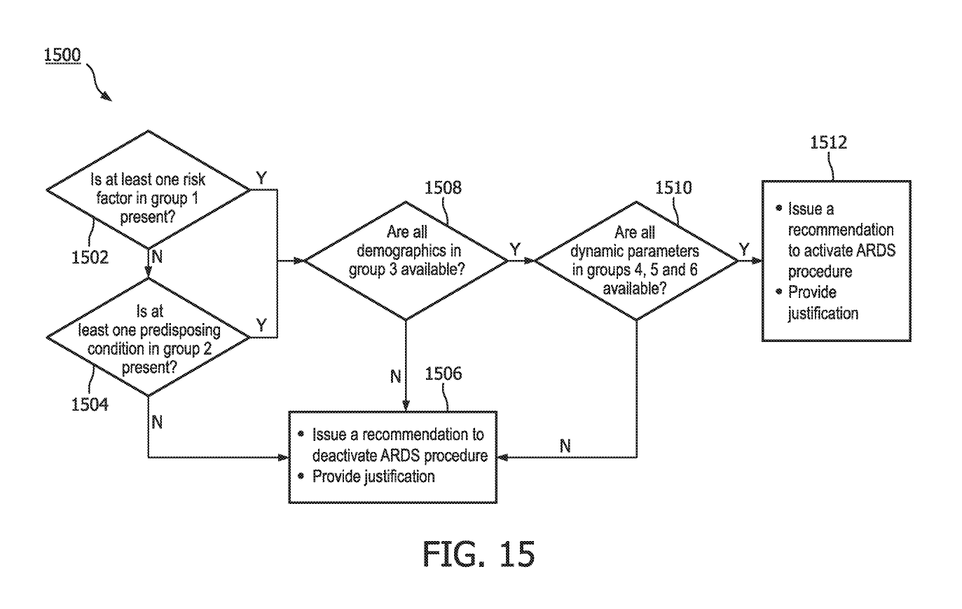

FIG. 15 depicts a flowchart of an exemplary method 1500 of providing clinical decision support in the context of monitoring Acute Respiratory Distress Syndrome (ARDS). ARDS is a complex response of the lung to direct or indirect insults characterized by sudden onset of severe hypoxemia (low oxygen levels in blood), radiographic evidence of bilateral pulmonary infiltration, and absence of left heart failure. Due to its complexity and lack of understanding of its development and progression, late symptom onset, delayed radiographic results, and the large number of risk factors and modifiers, only a fraction of ARDS patients are actually identified by bedside physicians.

Unidentified cases will develop various complications including multiple organ failures, which in turn can result in short-term or long-term disability, diminished quality of life and mortality, and excessive costs related to intensive care and rehabilitation. Thus, early diagnosis of ARDS will provide a wider therapeutic window for the prophylaxis and treatment of ARDS and its complications.

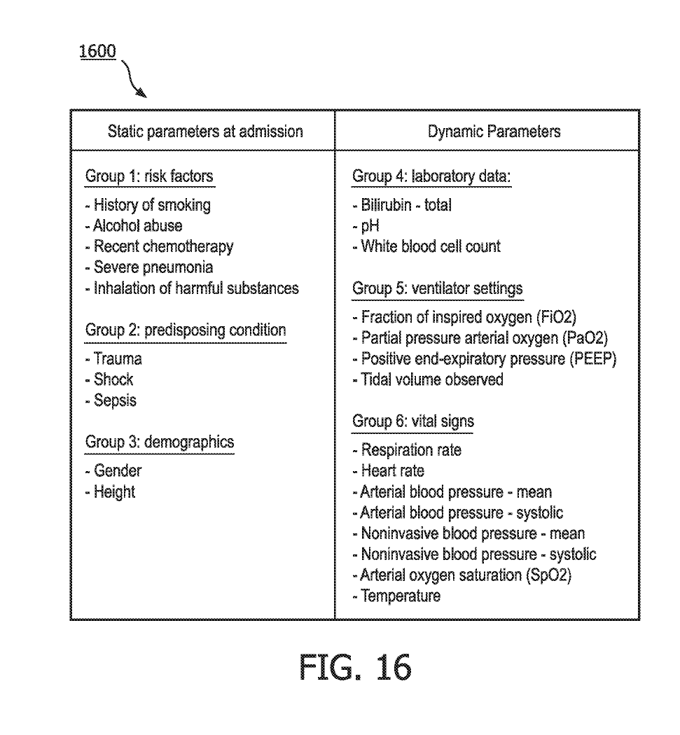

The method 1500 of FIG. 15 may assist in overcoming the above complications. Method 1500 of FIG. 15 is to be reviewed in conjunction with table 1600 of FIG. 16. Table 1600 lists exemplary parameters that may be considered when implementing the method 1500 of FIG. 15.

Step 1502 considers whether at least one risk factor in Group 1 (of table 1600 of FIG. 16) is present. Group 1 of table 1600 lists several risk factors for ARDS that may be known or otherwise obtained at the time of patient admission. These factors include, but are not necessarily limited to, history of smoking, alcohol abuse, recent chemotherapy, severe pneumonia, and inhalation of harmful substances.

If there is not at least one factor in Group 1 present, the method 1500 may proceed to step 1504. Step 1504 considers whether there is at least one predisposing condition in Group 2 of table 1600 present. Group 2 of table 1600 lists conditions that include, but are not necessarily limited to, trauma, shock, and sepsis.

If there is neither at least one factor from Group 1 present, nor at least one predisposing condition in Group 2 present, the method 1500 may proceed to step 1506. Step 1506 involves issuing a recommendation to deactivate the ARDS procedure, along with a justification for recommending deactivating the ARDS procedure. For example, the ARDS procedure may not be applicable due to insufficient information and/or none of the conditions indicative of ARDS are present.

If, on the other hand, at least one factor in Group 1 is present, at least one predisposing condition Group 2 is present, or both at least one factor in Group 1 and at least one predisposing condition in Group 2 is present, the method 1500 may proceed to step 1508. Step 1508 considers whether all demographics in Group 3 are available. The demographics in Group 3 of table 1600 include, but may not necessarily be limited to, patient gender and height. If not all demographics in Group 3 are available, the method 1500 may proceed to step 1506 to issue a recommendation to deactivate the ARDS procedure as discussed previously.

If all demographics in Group 3 are available, the method 1500 may proceed to step 1510. Step 1510 considers whether all dynamic parameters in groups 4, 5, and 6 are available. Groups 4, 5, and 6 include dynamic parameters relating to laboratory data, ventilator settings, and vital signs, respectively. The laboratory data of Group 4 may include, but is not necessarily limited to, the total amount of bilirubin, pH, and white blood cell count. The ventilator settings of Group 5 may include, but are not necessarily limited to, fraction of inspired oxygen, partial pressure arterial oxygen, positive end-expiratory pressure, and tidal volume observed. The vital signs of Group 6 include, but may not necessarily be limited to, respiration rate, heart rate, mean arterial blood pressure, systolic blood pressure, mean noninvasive blood pressure, systolic noninvasive blood pressure, arterial oxygen saturation, and temperature. If not all dynamic parameters of Groups 4, 5, and 6 are available, the method 1500 may proceed to step 1506 to issue a recommendation to deactivate the ARDS procedure as discussed previously.

If, on the other hand, all of the dynamic parameters of Groups 4, 5, and 6 are available, the method 1500 may proceed to step 1512. Step 1512 involves issuing a recommendation to activate the ARDS procedure. As can be deduced from steps 1502, 1504, 1508, 1510, and 1512, the recommendation to activate the ARDS procedure may be issued because at least one risk factor is present, at least one predisposing condition is present, all demographics are available, and all laboratory data is available.

The methods described above may be performed either at predefined run times (e.g., every 15 minutes) or whenever it is triggered by an input (e.g., a dynamic parameter). With reference to FIG. 15, for example, one embodiment could involve recommending activation of the ARDS procedure when a high risk of ARDS such as sepsis or shock is internally detected. This may help avoid late detection of ARDS if the procedure is not activated because of invisible symptoms or other critical conditions.

It is also noted that the steps of the methods described above may vary, and may be performed in different orders than illustrated and described above. For example, rather than considering whether information is merely present (e.g., arterial oxygen saturation of table 1600 in step 1510), method 1500 may proceed to step 1512 only if the arterial oxygen saturation is at or below a predetermined threshold level.

In some embodiments, various derivatives of the measured parameters may be calculated, and used to decide whether or not to change the activation status of a procedure. For example, these derivatives may include, but are not limited to, simple averages (i.e., mean(s)), weighted averages, standard deviations, etc. Determining whether or not to change the activation status of a procedure may be based on these calculated derivatives.

Additionally, and as mentioned previously, methods 900 and/or 1100 may automatically change the activation status of the respective procedure rather than just issuing a recommendation to activate or deactivate a procedure. In this scenario, a notification regarding the change in activation status may be issued, along with a justification as to why the activation status was changed.

Methods 900, 1100, 1300, and 1500 are merely exemplary procedures. It is contemplated that the features of the invention may be extended to other procedures relating to other health conditions of a patient.

For example, Applicants have developed an Early Deterioration Index (EDI) procedure that uses a plurality of vital signs (systolic blood pressure, heart rate, respiratory rate, and temperature, age, and O₂ saturation) to develop a single value representing probability of deterioration. It may be used as part of a “track-and-trigger” system where an increasing score triggers escalated responses, varying from increasing frequency of observations to calling for a review by a rapid response team. It has also been proposed that the EDI score can be used as a measure of patient acuity.

In this example, the EDI score can be calculated for all patients when vital signs are captured and the score can be sent to the processor 102 of FIG. 1. This particular procedure could have a minimum score threshold (e.g., 20%) at which point it recommends that an operator activate the EDI procedure for display and trending. When scores are below the threshold, the EDI procedure may appear to be deactivated, thus reducing the amount of data that needs to be presented to the operator.

In a related example, assume one of the vital signs used in the EDI calculation (e.g., respiration rate) is not present. The EDI score may nonetheless be calculated using only the remaining parameters. If the EDI score is high, in this case, the processor 102 could inform an operator, via the user interface 112, that the EDI score is high and recommend that the EDI procedure be visibly activated and the missing vital sign (i.e., respiration rate) be measured. If the resulting EDI score remains high with the addition of the missing parameter, the processor 102 may recommend that the EDI procedure remain activated and its score be observed.

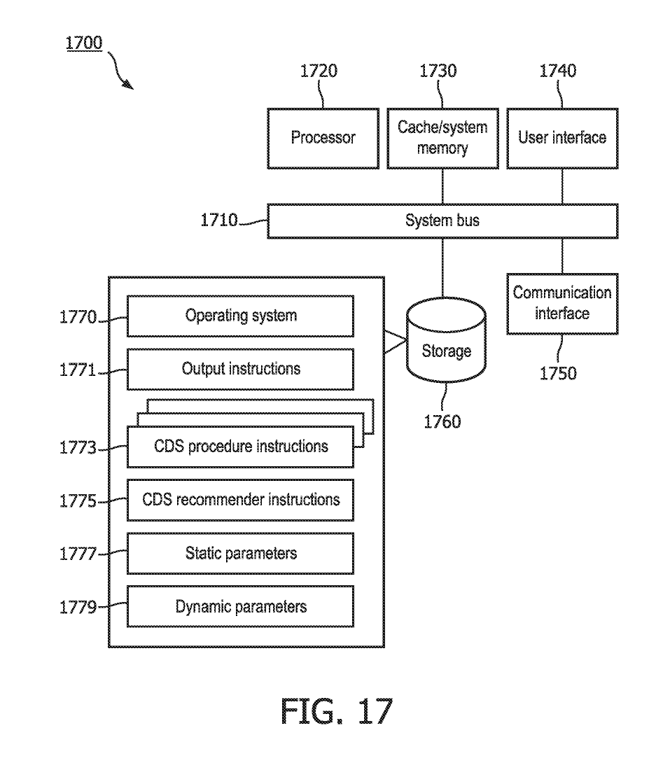

FIG. 17 illustrates an example of a hardware system 1700 for implementing various devices that may participate in the various methods described herein. The hardware 1700 may implement a user mobile device or a supporter mobile device. As shown in FIG. 17, the hardware 1700 includes one or more system buses 1710 that connect a processor 1720, cache/system memory 1730, a user interface 1740, a communication interface 1750, and storage 1760. It will be understood that FIG. 17 is merely exemplary and constitutes, in some respects, an abstraction and that the actual organization of the components of the hardware 1700 may vary and be more complex than illustrated.

The processor 1720 may be any hardware device capable of executing instructions stored in memory 1730 or storage 1760 or otherwise processing data. As such, the processor 1720 may include a microprocessor, a field programmable gate array (FPGA), application-specific integrated circuit (ASIC), or other similar devices. In some embodiments, such as those relying on one or more ASICs, the functionality described as being provided in part via software may instead be hardwired into the operation of the ASICs and, as such, the associated software may be omitted.

The cache/system memory 1730 may include various memories such as, for example, L1, L2, or L3 cache or system memory. As such, the memory 1730 may include static random access memory (SRAM), dynamic RAM (DRAM), flash memory, read only memory (ROM), or other similar memory devices.

The memory 1730 may be pre-configured to store a set of procedures to be run by the processor 1720. Or, the memory 1730 may routinely be updated to store certain procedures. The procedures stored by the memory 1730 may vary and may depend on the particular patient.

The user interface 1740 may include one or more devices for enabling communication with a user such as an operator. For example, the user interface 1740 may include a display, a mouse, a keyboard, a touchscreen, buttons, camera, microphone, vibrator, haptic engine, etc. In some embodiments, the user interface 1740 may include a command line interface or graphical user interface that may be presented to a remote terminal via the communication interface 1750.

The communication interface 1750 may include one or more devices for enabling communication with other hardware devices. For example, the communication interface 1750 may include a network interface card (MC) configured to communicate according to WiFi or Ethernet protocols. Additionally the communication interface 1750 may implement a TCP/IP stack for communicating according to the TCP/IP protocols. In some embodiments, the communication interface 1750 may include an NFC, Bluetooth, or other short range wireless interface. Various alternative or additional hardware or configurations for the communication interface 1750 will be apparent.

The storage 1760 may include one or more machine-readable storage media such as read-only memory (ROM), random-access memory (RAM), magnetic disk storage media, optical storage media, flash-memory devise, or similar storage media. In various embodiments, the storage 1760 may store instructions for execution by the processor 1720 or data upon which the processor 1720 may operate. For example, the storage 1760 may store an operating system 1770 for controlling various basic operations of the hardware system 1700. The storage 1760 may also include output instructions 1771 for rendering a display (e.g., a graphical user interface also capable of receiving user input) of the outputs of various CDS procedure instructions 1773. CDS recommender instructions may implement one or more of the aforementioned methods or methods similar thereto for processing static parameters 1777, dynamic parameters 1779, and/or outputs from the CDS procedure instructions 1773 to determine when to activate/deactivate (or recommend activation/deactivation via the interface created by the output instructions 171 of) one of more of the CDS procedure instructions 1773.

The methods, systems, and devices discussed above are examples. Various configurations may omit, substitute, or add various procedures or components as appropriate. For instance, in alternative configurations, the methods may be performed in an order different from that described, and that various steps may be added, omitted, or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims.

Embodiments of the present disclosure, for example, are described above with reference to block diagrams and/or operational illustrations of methods, systems, and computer program products according to embodiments of the present disclosure. The functions/acts noted in the blocks may occur out of the order as shown in any flowchart. For example, two blocks shown in succession may in fact be executed substantially concurrent or the blocks may sometimes be executed in the reverse order, depending upon the functionality/acts involved. Additionally, or alternatively, not all of the blocks shown in any flowchart need to be performed and/or executed. For example, if a given flowchart has five blocks containing functions/acts, it may be the case that only three of the five blocks are performed and/or executed. In this example, any of the three of the five blocks may be performed and/or executed.

A statement that a value exceeds (or is more than) a first threshold value is equivalent to a statement that the value meets or exceeds a second threshold value that is slightly greater than the first threshold value, e.g., the second threshold value being one value higher than the first threshold value in the resolution of a relevant system. A statement that a value is less than (or is within) a first threshold value is equivalent to a statement that the value is less than or equal to a second threshold value that is slightly lower than the first threshold value, e.g., the second threshold value being one value lower than the first threshold value in the resolution of the relevant system.

Specific details are given in the description to provide a thorough understanding of example configurations (including implementations). However, configurations may be practiced without these specific details. For example, well-known circuits, processes, algorithms, structures, and techniques have been shown without unnecessary detail in order to avoid obscuring the configurations. This description provides example configurations only, and does not limit the scope, applicability, or configurations of the claims. Rather, the preceding description of the configurations will provide those skilled in the art with an enabling description for implementing described techniques. Various changes may be made in the function and arrangement of elements without departing from the spirit or scope of the disclosure.

Having described several example configurations, various modifications, alternative constructions, and equivalents may be used without departing from the spirit of the disclosure. For example, the above elements may be components of a larger system, wherein other rules may take precedence over or otherwise modify the application of various implementations or techniques of the present disclosure. Also, a number of steps may be undertaken before, during, or after the above elements are considered.

Having been provided with the description and illustration of the present application, one skilled in the art may envision variations, modifications, and alternate embodiments falling within the general inventive concept discussed in this application that do not depart from the scope of the following claims.

1. A method of providing clinical decision support for at least one patient, the method comprising:
obtaining medical data for the at least one patient;
analyzing, using a processor, the medical data using a plurality of CDS procedures, wherein each of the plurality of CDS procedures has an activation status and utilizes the medical data to make a recommendation concerning a clinical procedure; changing, using the processor, the activation status of a CDS procedure from deactivated to activated based on the analysis of the medical data; and
supplying the recommendation concerning a clinical procedure from the activated CDS procedure using an interface.

2. (canceled)

3. (canceled)

4. The method of claim 1, further comprising changing, using the processor, the activation status of the activated CDS procedure from activated to deactivated in response to insufficient data related to the at least one patient.

5. The method of claim 1, further comprising changing, using the processor, the activation status of the activated CDS procedure from activated to deactivated by stopping the procedure from analyzing the obtained data.

6. The method of claim 1, wherein the data for the at least one patient includes one or more of patient history, patient demographics, patient symptoms, patient diagnosis, patient vital signs, laboratory data, and patient current health status.

7. The method of claim 1, wherein effecting the change of the activation status of the CDS procedure from deactivated to activated comprises automatically activating the CDS procedure.

8. The method of claim 1, wherein changing the activation status of the CDS procedure from deactivated to activated comprises issuing a recommendation to change the activation status of the CDS procedure to at least one operator before changing the activation status of the CDS procedure.

9. The method of claim 1, further comprising determining, using the processor, a relevancy of the CDS procedure, wherein the processor changes the activation status of the CDS procedure based on the determination.

10. (canceled)

11. An apparatus for providing clinical decision (CDS) support for at least one patient, the apparatus comprising:
a memory; and
a processor in operable communication with the memory, wherein the processor is configured to:
analyze patient medical data using a plurality of CDS procedures, wherein each of the plurality of CDS procedures has an activation status;
changing the activation status of a CDS procedure from deactivated to activated based on the analysis of the medical data; and
supplying the recommendation concerning a clinical data procedure from the activated CDS procedure using an interface.

12. (canceled)

13. (canceled)

14. The apparatus of claim 11, wherein the processor is further configured to change the activation status of the activated CDS procedure from activated to deactivated in response to insufficient data related to the at least one patient.

15. The apparatus of claim 11, wherein the processor is further configured to change the activation status of the activated CDS procedure from activated to deactivated by stopping the procedure from analyzing the patient medical data.

16. The apparatus of claim 11, wherein the patient medical data includes one or more of patient history, patient demographics, patient symptoms, patient diagnosis, patient vital signs, laboratory data, and patient current health status.

17. The apparatus of claim 11, wherein in effecting the change of the activation status of the CDS procedure from deactivated to activated the processor is configured to automatically activate the CDS procedure.

18. The apparatus of claim 11, wherein in effecting the change of the activation status of the CDS procedure from deactivated to activated the processor is configured to issue a recommendation to change the activation status of the CDS procedure to at least one operator before changing the activation status of the CDS procedure.

19. The apparatus of claim 11, wherein the processor is further configured to determine a relevancy of the CDS procedure, wherein the processor changes the activation status of the CDS procedure based on the determination.

20. (canceled)
